(19) United States
(12) Patent Application Publication
(10) Pub. No.: US 2007/0213775 A1
(43) Pub. Date: Sep. 13, 2007

(54) External Defibrillator With Pre-Cpr-Ecg Based Defibrillating Shock

(75) Inventor: David E. Snyder, Bainbridge Island, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/573,103

(22) PCT Filed: Aug. 9, 2005
(86) PCT No.: PCT/IB05/52420

§ 371 Date: Feb. 2, 2007

(30) Foreign Application Priority Data
Jul. 19, 2005 (IB) PCT/IB05/52420

(51) Int. Cl. A61N 1/39

(52) U.S. Cl. 607/7

(57) ABSTRACT

A defibrillator having a pair of electrodes for delivering an artifact-compensated defibrillation shock and a method thereof is provided. Electrocardiographic (ECG) data representative of an ECG signal is acquired for the patient and the ECG data is analyzed to determine whether administration of a defibrillating shock is appropriate. A cardio-pulmonary resuscitation (CPR) interval is initiated subsequent to the acquisition of ECG data. Upon completion of the CPR interval, and if administration of a defibrillating shock is determined to be appropriate from the ECG data, arming the AED for delivery of the defibrillation shock to the patient.

The present invention relates to devices used in electrical therapy and, in particular, to a defibrillator for rapidly delivering defibrillation pulses with minimal delay following intervals of cardio-pulmonary resuscitation (CPR) performed on a patient.

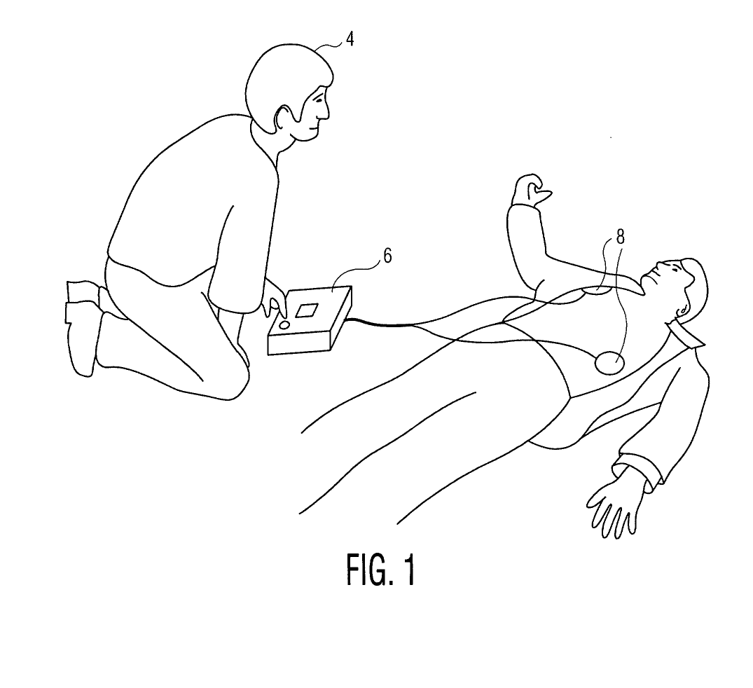

Medical equipment manufacturers have developed Automated Electronic Defibrillators (AEDs) to provide early defibrillation. AEDs deliver a high-amplitude current pulse, waveform, or shock to the heart in order to restore the patient's heart rhythm to a normal level. For example, FIG. 1 depicts the conventional AED 6 being applied to a cardiac arrest victim 2 by a rescuer 4. As shown in FIG. 1, a pair of defibrillation electrodes 8 is placed on anterior-anterior (AA) positions on the victim's torso for delivering the shocks. It is often necessary to perform a cardio-pulmonary resuscitation (CPR) on the patient interspersed with defibrillation shocks in order to revive the victim from the cardiac arrest.

In treating victims of cardiac arrest with a defibrillator, it is important that the treatment be performed very rapidly as their chances of surviving the cardiac arrest decrease drastically over time following the cardiac arrest. Thus, a quick response to cardiac arrest in administering a first defibrillation shock from the beginning of the arrest is important. Also, when CPR precordial compressions are performed at the rescue scene to improve the chance of survival, a long pause between discontinuation of CPR on a patient experiencing ventricular fibrillation and shock delivery will markedly decrease the chance of survival. Thus, it is critical to shorten the process of analyzing the heart rhythm before or immediately following the discontinuation of the CPR in order to rapidly arm the AED to deliver a subsequent defibrillation shock.

Therefore, the present invention provides an improved defibrillator that is easy to use and that enables a minimally trained user to easily, rapidly, and effectively deploy the defibrillator to treat the patient, while reducing the time interval between precordial compressions and delivery of a defibrillation shock.

The present invention is directed to a method and system for quickly and accurately applying the defibrillating shock to a victim of sudden cardiac arrest, especially following delivery of CPR precordial compressions.

According to one aspect of the invention, a delay between the administration of CPR and the delivery of defibrillation shock is minimized by quickly discriminating the end of a period of CPR and charging the defibrillator.

According to another aspect of the invention, a method of applying electrotherapy in an automatic external defibrillator (AED) of the type having a high voltage energy source, an ECG detector, and a CPR therapy module is provided. In this method, an indication of CPR cessation is obtained in order to arm the AED for a subsequent electrotherapy shock. The charging of the AED may be initiated prior to the end of the CPR therapy interval or completed prior to the end of the CPR therapy interval. The method further includes obtaining an ECG signal from the ECG detector prior the end of the CPR therapy interval, and determining whether the ECG signal is corrupted by CPR activity.

According to another aspect of the invention, a defibrillator having a CPR delivery system is provided and includes: a detector arranged to detect a signal indicating the cessation of CPR; an energy source for providing the defibrillation shock energy; a charging circuit for charging the energy source; and a controller for controlling the charging circuit responsive to the signal. The interval between the cessation of CPR to the defibrillator being charged for delivery of the defibrillation shock is less than about 10 seconds. The defibrillator further includes an ECG detector for detecting an ECG rhythm signal, so that processor may charge the defibrillator responsive to a detected shockable ECG rhythm.

According to yet another aspect of the invention, an apparatus for delivering a defibrillation shock to a patient is provided. The apparatus includes at least one sensor adapted to contact the patient; a detector coupled to the sensor for detecting an input signal indicative of disturbances associated with the application of cardiopulmonary resuscitation; a processor for receiving the input signal from the detection system, for analyzing the detected input signal to produce a signal indicative of corruption of electrocardiographic (ECG) signals from the patient, and for determining if a defibrillation shock is needed; and a discharger for providing defibrillation shocks to a patient. The apparatus further includes an ECG front end coupled to the pair of electrodes to determine the patient impedance, an LCD display, and a speaker to notify an operator prior to discharging the defibrillation shock.

Still another aspect of the invention provides a method for delivering a defibrillation shock to a patient using a defibrillator. The method includes charging the defibrillator prior to end of a cardio-pulmonary resuscitation (CPR) interval; analyzing an ECG signal prior to end of a cardio-pulmonary resuscitation (CPR) interval; and delivering a defibrillation shock after the cardio-pulmonary resuscitation (CPR) interval if there is no signal corruption associated with the administration of CPR.

According to another aspect of the invention, a method for delivering a defibrillation shock to a patient from a defibrillator is provided. The method includes acquiring ECG data representative of an ECG signal for the patient and analyzing the ECG data to determine whether administration of a defibrillating shock is appropriate. A CPR interval is initiated subsequent to the acquisition of ECG data. In response to the completion of the CPR interval, and if administration of a defibrillating shock is determined to be appropriate from the ECG data, arming the AED for delivery of the defibrillation shock to the patient.

In the drawings:

FIG. 1 is an illustration of a defibrillator being applied to a patient under cardiac arrest according to an embodiment of the present invention;

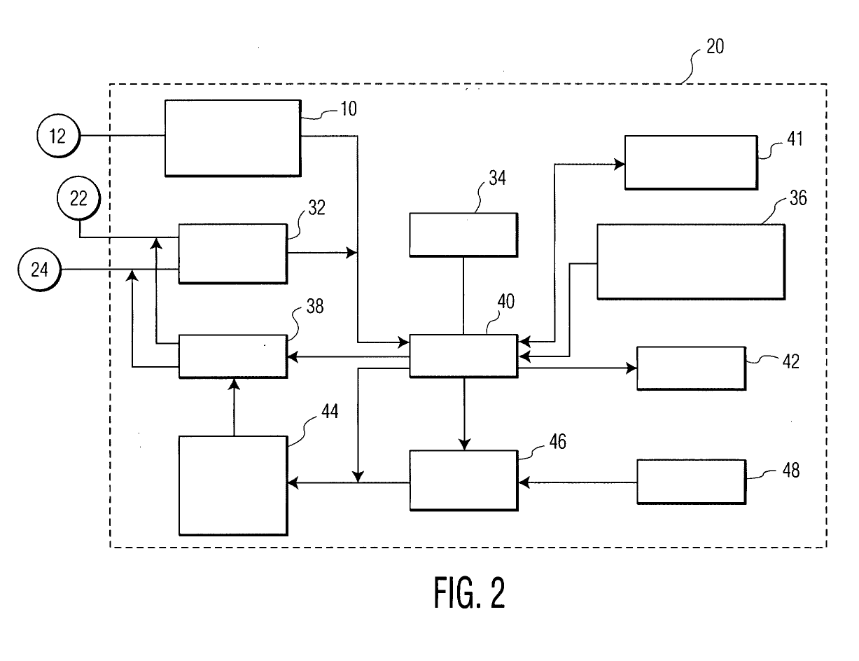

FIG. 2 depicts a representative hardware of the defibrillator illustrated in FIG. 1 according to an embodiment of the present invention;

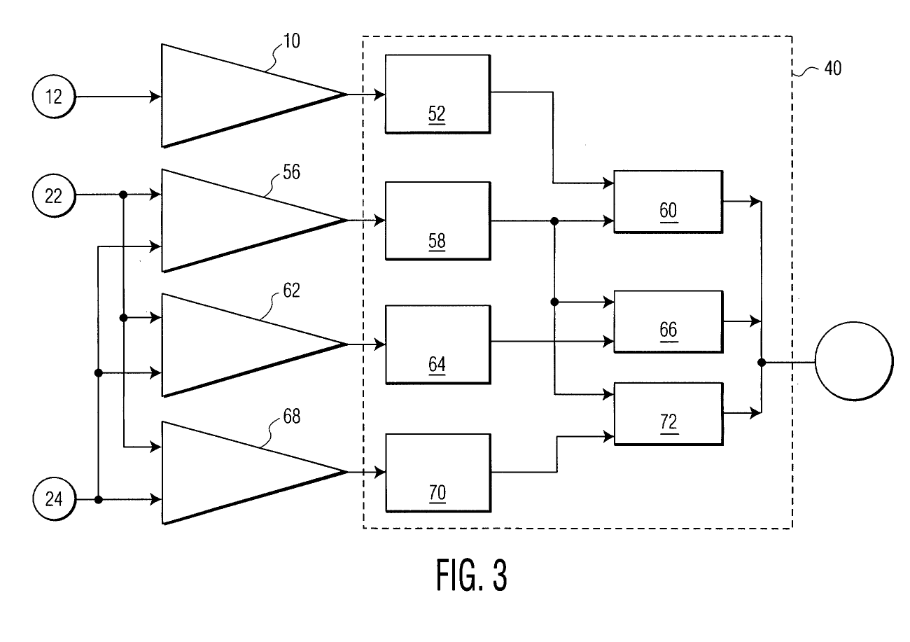

FIG. 3 is a diagram of the hardware configured to deliver a defibrillation shock according to an embodiment of the present invention;

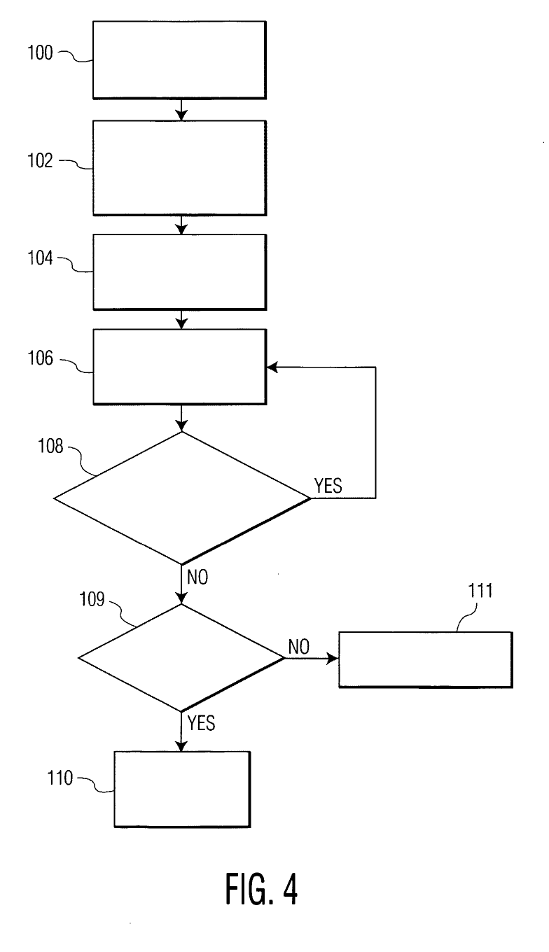

FIG. 4 is a flow chart illustrating the operation steps of the defibrillation system in accordance with an embodiment of the present invention;

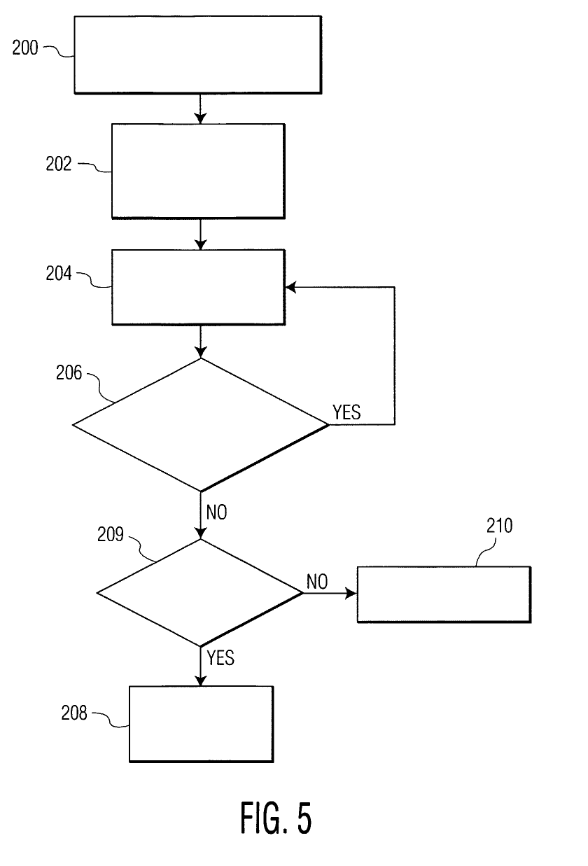

FIG. 5 is a flow chart illustrating the operation steps of delivering a defibrillation shock according another embodiment of the present invention;

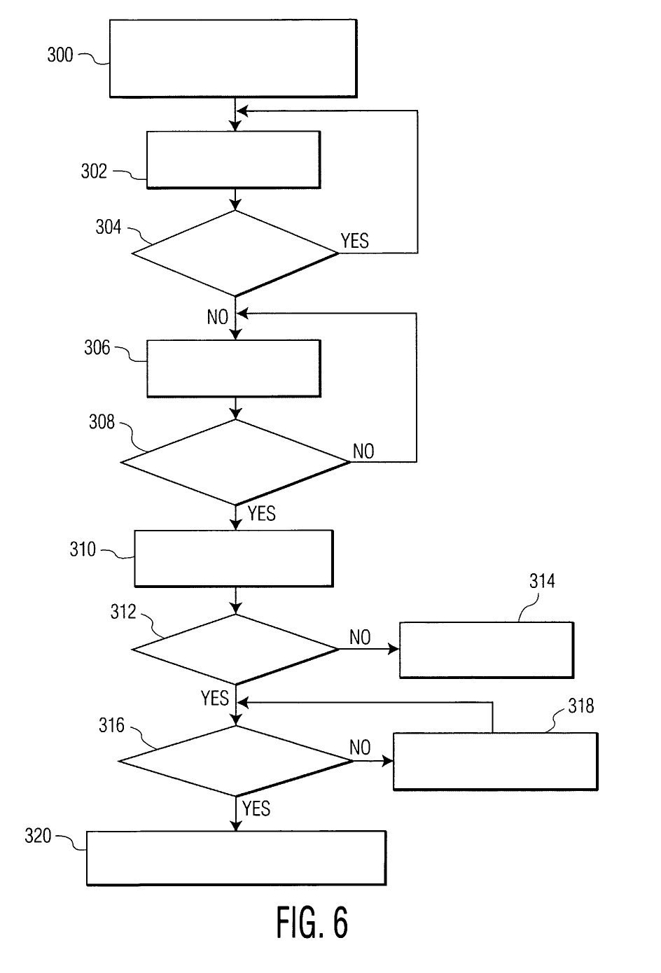

FIG. 6 is a flow chart illustrating the operation steps of delivering a defibrillation shock according another embodiment of the present invention; and

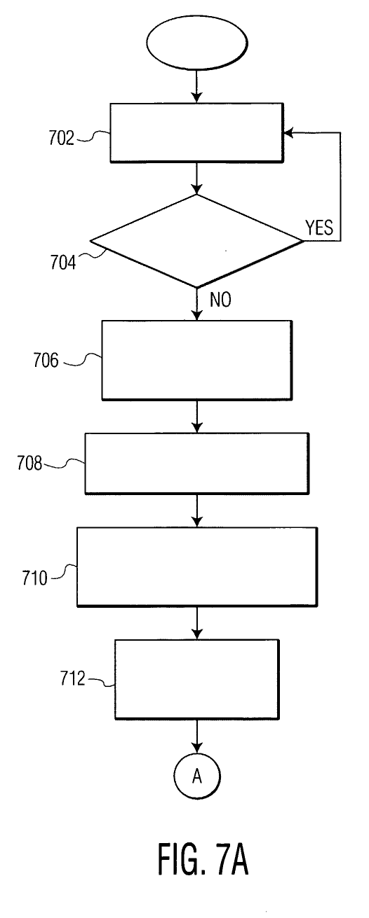

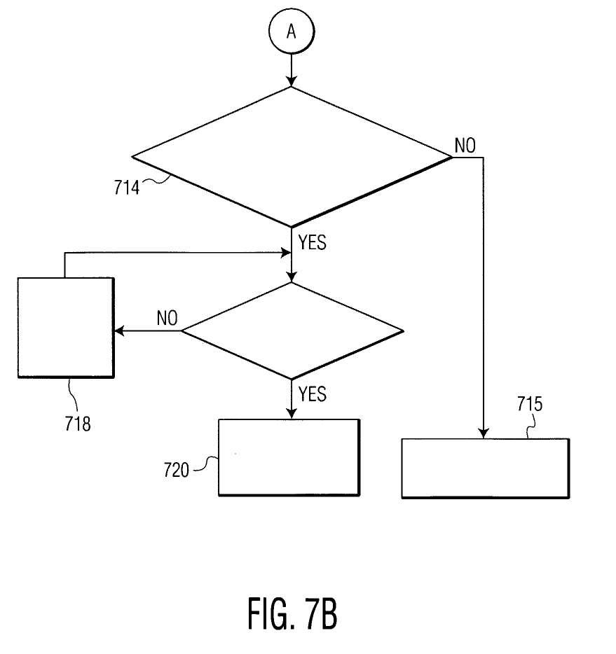

FIG. 7 is a flow chart illustrating the operation steps of delivering a defibrillation shock according another embodiment of the present invention.

In order to facilitate an understanding of this invention, a conventional method of providing resuscitation of a victim will be described.

During the course of a resuscitation of a victim in sudden cardiac arrest, a rescuer often follows a protocol that calls for the application of defibrillation shocks from an Automated Electronic Defibrillator (AED) and delivering intervals of cardiopulmonary resuscitation (CPR) to promote the circulation of blood until normal circulation can be re-established. In many cases, the resuscitation protocol is directed to the rescuer via the AED using voice prompting. For example, a victim of cardiac arrest due to ventricular fibrillation may receive several defibrillation shocks followed by a preprogrammed interval of CPR, during which the rescuer provides precordial compressions to the victim plus breathing assistance.

As one of average skilled in the art will appreciate, precordial compressions during CPR are thought to provide artificial circulation, which improves chances of survival, by the victim. Following a predetermined interval of precordial compressions, the operator is typically prompted to stop CPR so that the AED may analyze the victim's ECG rhythm to determine if more shocks are needed. However, it has been discovered that the likelihood of survival rapidly falls as the time interval between cessation of compressions and the delivery of a defibrillation shock increases. It is therefore important to minimize this critical interval as much as possible, preferably to less than ten seconds.

In conventional systems, four steps must occur following a CPR interval before an AED can deliver a shock: (1) The prescribed CPR time interval set by the rescue protocol must expire (one to three minutes, typically); (2) The patient's ECG rhythm must be analyzed to determine if the rhythm should be shocked; (3) The AED must fully charge its energy storage capacitor; and (4) The AED must arm to deliver the shock.

In association with these steps, AEDs typically issue voice prompts to the operator at the end of a CPR sequence in order to advise the operator not to touch the patient, which may interfere with and thus delay analysis of the patient's ECG rhythm. Following AED analysis and arming to shock, the operator is often again warned not to touch the patient and to deliver the shock by pressing a SHOCK button (if the device is semiautomatic) or to warn that a shock will be delivered (if the AED is fully automatic).

In most prior art devices, the four stages above proceed in serial sequence, which results in a period of typically 15 to 30 seconds or more between the end of precordial compressions and the possible delivery of a shock to a patient. In some cases, prior art devices partially charge the AED's energy storage capacitor to save some time prior to the completion of ECG analysis, but do not fully charge until analysis is completed. Some prior art AEDs employ artifact detection during attempts to analyze patient's ECG signals in order to determine if a shock should be delivered. Artifact detection acquires a disturbance signal, which can originate from patient motion or any other possible source of ECG disturbance (e.g. electromagnetic), and compares it to the patient's ECG, e.g. by cross-correlation, in order to determine if the disturbance has manifested itself as corruption of the ECG. Such a corruption would make analysis of the ECG for shock advisory purposes unreliable as the ECG is known to contain signals that are not of cardiac origin. Generally, these artifact detection systems have been used to inhibit the analysis of corrupted or potentially corrupted ECG signals and to prompt the operator to remove the source of interference.

Furthermore, prior art defibrillators have presented a shock hazard to the rescuer as the defibrillators are incapable of detecting whether the rescuer was in physical contact with the patient. One way to address this problem in the prior is to provide a “no touch” interval period prior to delivering the therapy shocks. However, this delay is undesirable as it delays the interval between the CPR precordial compressions, if occasion demands, and the delivery of defibrillation shock, thus preventing a quick response in administering a defibrillation shock which is vital in increasing the chance of surviving the cardiac arrest.

In contrast, one embodiment of the present invention provides a defibrillation system in which the delivery of electrical therapy shocks is triggered by a combination of the detection of a treatable arrhythmia via an ECG analysis and a detection of the cessation or absence of CPR precordial compressions. In accordance with this embodiment, the defibrillation system reduces the time interval between precordial compressions and a subsequent delivery of the defibrillation shock.

FIG. 2 is a simplified block diagram of a defibrillator 20 in accordance with this embodiment of the present invention. The defibrillator 20 may include a mechanical disturbance detector 10, an electrocardiogram (ECG) front end 32, a timer 34, a defibrillation activation/deactivation button 36, a high voltage (HV) switch 38, a processor 40, a voice circuit and speaker 41, a display 42, an energy storage capacitor network 44, a voltage charger 46, and a battery 48. While particular reference is made to the system block diagram of FIG. 1, it is to be understood at the outset of the description which follows, that is contemplated that the apparatus and methods in accordance with embodiments of the present invention may be used with other hardware configurations of the planner board. Further, it should be noted that any number of commercially or publicly available defibrillators configured to generate a defibrillation shock can be utilized in various implementations in accordance with the preferred embodiment of the present invention.

As shown in FIG. 2, the mechanical disturbance detector 10 is connected to a sensor 12 that is placed on the patient to detect the movement of the patient during the delivery of CPR precordial compressions. The movement of the patient that may potentially corrupt the accurate assessment of the signal of interest is detected and forwarded to the processor 40. Similarly, the ECG front end 32 is connected to the electrodes 22 and 24 that are placed on the patient to amplify, filter, and digitize (using an analog to a digital converter) an electrical ECG signal generated by the patient's heart. The detected ECG samples are received by the processor 40, which runs a shock advisory algorithm for detecting VF or other shockable rhythm requiring treatment by the defibrillation shock. The ECG front end 32 is also capable of measuring the patient impedance across the electrodes 22 and 24 using a low-level test signal that is a non-therapeutic pulse to measure the voltage drop across the electrodes 22 and 24. In an alternate embodiment, the function of the mechanical disturbance detector 10 and the ECG front end 32 can be merged as one component, such that one of the electrodes connected to the ECG front end 32 may serve as a sensor for detecting the movement of the patient.

The HV switch 38 is configured to sequentially deliver the defibrillation pulse across the pair of electrodes 22 and 24 to the patient in the desired polarity and duration. It should be noted that the HV switch 38 could be adapted to deliver a single polarity (monophasic), both negative and positive polarities (biphasic) or multiple negative and positive polarities (multiphasic) in the preferred embodiment. The timer 34 is connected to the processor 40 for providing a defibrillation pulse interval or duration when delivering the defibrillation pulse across the electrode pair 22 and 24. The activation/deactivation button 36 is connected to the processor 40 to enable the user to activate/deactivate the delivery of a defibrillation pulse across the electrodes 22 and 24 when the VF or other shockable rhythm is detected. Note that the activation/deactivation button 36 can function in both AED and manual modes in the preferred embodiment. The voice circuit/speaker 41 provides voice instructions to the user during the operation of the defibrillator 20. Alternatively, in other embodiments such as a fully automated AED the activation/deactivation button may be omitted. The display 42, connected to the processor 40, is preferably a liquid crystal display (LCD) and provides visible feedback to the user. The battery 48 provides power for the defibrillator 20 and in particular for the charger 46, which charges the capacitors in the energy-storage capacitor network 44. The energy-storage capacitor network 44 includes a plurality of capacitors and resistors that are arranged in series or parallel arrangement, or a combination of series and parallel arrangement to supply a plurality of voltage-level outputs across the electrodes 22 and 24. It will be apparent to those skilled in the art that a variety of RC arrangements can be implemented to generate different defibrillation pulse characteristics.

In operation, the electrodes 22 and 24 connected to the ECG front end 32 are placed on a patient for obtaining the patient impedance. The defibrillation pulse delivered to the patient may be a fixed level, or a number of defibrillation pulses at different energy levels. This can be achieved by selecting the appropriate voltage level of the energy-storage capacitor network 44 from the set of configurations to deliver the desired impedance-compensated defibrillation pulse to the patient.

When a rescuer performs chest compressions as part of doing CPR on the patient, the resulting chest movement tends to disturb the electrodes placed on the chest area. This is undesirable for detecting ECG signals as the movement of the electrodes on the chest skin area generates interfering electrical noise or artifacts, which may corrupt the ECG signal. As discussed in detail later, the artifact in the ECG signal caused by mechanical disturbances of sensors, electromagnetic interference, other environmental conditions, or artifact caused by movement due to the cardiopulmonary resuscitation (CPR) operation are nevertheless useful as indicators that CPR is being performed, when CPR is concluded, and when the patient is being handled. An accelerometer or other motion sensor may be included in some embodiments for such detection purposes. The defibrillator 20 is also provided with the display 42 for visually indicating the shock annunciation, or may be equipped with a voice circuit and speaker 41 for providing audible announcement just prior to delivering the defibrillation shocks.

Note that during the rescue attempt, if renewed mechanical disturbance and/or artifact is detected after the AED arms for a shock, the defibrillation activation/deactivation 36 is provided for cancellation of the shock within the brief delay interval prior to therapy delivery. Alternatively, the motion of new mechanical disturbance and/or artifact can be detected automatically and cause the cancellation or delay of the defibrillation shock therapy.

FIG. 3 is a detailed description of the components that enable a rapid analysis during mechanical disturbances, so that arrhythmia determination can be made using artifact-free ECG, especially following the cessation of mechanical disturbance. Note that detecting artifact can be performed in a variety of ways. For example, signal processing and correlation of processed signals in accordance with the present invention may include various embodiments described in U.S. Pat. No. 6,287,328, filed by the applicant and issued on Sep. 11, 2001, entitled “Multivariable Artifact Assessment,” the teachings of which are incorporated herein by reference.

Briefly, an input signal indicative of patient movement is received via the sensor 12 and provided to the measurement circuit 10. The signal is then sent to a signal processor 52 and forwarded to a correlator 60 for correlation with the patient's ECG signal, and the correlated signal is transmitted to the processor 40. As will be appreciated by those skilled in the art, an appropriate signal processing includes, for example, band-pass filters, Fourier transforms, wavelet transforms, time domain analysis, or joint time-frequency spectrograms. In addition, the method for correlating the data can be any correlation method known in the art. For example, correlation methods include specific and general cross-correlation techniques, which include known mathematical functions as well as any process that effectively correlates the data. Specific implementations include, but are not limited to, finite sampled or continuous estimates of cross-covariance and cross-correlation, both biased and unbiased. Alternatively, correlation may perform similarity comparisons between any multiple signals.

Meanwhile, an input signal indicative of the patient voltages and impedance is received across the electrodes 22 and 24 and transmitted to a differential-mode amplifier 56 and a common-mode amplifier 62 which amplify the signal prior to transmitting the signal to the signals processors. The resulting signals are then transmitted to their respective signal processors 58 and 64 which process the signals to emphasize particular features. Specifically, signal processor 58 supplies signal information for correlation with variables including common-mode signals, impedance signals, or motion signals from their respective processors. The resulting processed signals are then transmitted to a correlator 66, which correlates the signals. At the same time, the input signal is transmitted to impedance detector 68, which provides a trans-electrode impedance signal to the signal processor 70. Signal processor 70 processes the signal from the impedance detector 68 to emphasize particular features of the signal. The resulting processed signal is then transmitted to a correlator 72, which correlates the signal processor 70 with the processed signal from the differential amplifier 58. Once the signals have been correlated at their respective correlators 66, 72, the resulting signals are transmitted to the processor 40, which then further evaluates the results of the correlators 66 and 72 to provide an indication of the degree of corruption of the ECG signal of interest. The processor 40 finally provides an output signal, which may be analyzed further as discussed with respect to FIG. 4 below.

FIG. 4 is a flow chart illustrating the operation steps of delivering an artifact-compensated defibrillation shock according to a preferred embodiment of the present invention.

Initially, the defibrillator 20 is deployed by attaching the sensor 12 and the electrodes 22, 24 to the cardiac victim to analyze a patient input signal. Note that it is common to perform CPR including precordial compressions on the victim in conjunction with the use of the defibrillator during rescue attempts, so possibly ongoing CPR is detected by the input signal from the sensor 12.

In step 100, the system 20 begins to charge the capacitor 44 to an intermediate level at a predetermined interval prior to the end of the CPR precordial compression. In step 102, the system 20 sends a message to the rescuer to stop the CPR, during which the capacitor is still being charged. A short interval, for example 3 seconds or less, is allowed in step 104. Thereafter, the electrodes 22 and 24 connected to the ECG front end 32 detect an input signal, i.e., ventricular fibrillation (VF) and the patient impedance, measured by measuring a low-level test signal or delivering a non-therapeutic signal in step 106. Motion information is acquired by motion disturbance detector 10 or motion sensor.

In step 108 the ECG signal is tested against the other measurements in order to determine if the ECG signal is being corrupted. The signal processing is implemented in order to emphasize a particular feature of the data in the input signal. Here, various implementations of processing, including known techniques such as filters, Fourier transforms, wavelet transforms, or joint time-frequency spectrograms, are employed. For example, the lower spectral portion of a Fourier transform of an ECG signal might be correlated with a similarly processed impedance signal in order to enhance the detection of an artifact resulting from a defibrillator operator performing CPR on a patient being monitored.

Accordingly, the analyzing step 108 performs the function of measuring similarities between the processed cardiac signal and the processed corrupted signals. The resulting comparisons are then analyzed to determine an indication of the amount of artifacts present within the potentially corrupted cardiac signal. If artifact and/or motion are detected in step 108, the process returns to step 106.

Once step 108 determines that acquired ECG data is not corrupted the ECG data is analyzed by processor 40 to determine if a shock is required in step 109. If a shock is not required, the AED may provide alternate care instructions to the rescuer. If the ECG rhythm should be shocked, however, the AED completes its capacitor charge in step 110. Further, absence of either ECG corruption or motion disturbance signals in step 108 indicates that the rescuer has discontinued CPR, and it is therefore appropriate to immediately arm the defibrillator to deliver a shock. Processor 40 then sends a signal to the HV switch 38 to actuate the switches to discharge the desired defibrillation shock to the patient. Alternatively, the processor 40 may notify the operator via the display 42 to press the shock button 36 to actuate manually the delivery of the defibrillation shock to the patient. Accordingly, the defibrillation shock is discharged to the patient in step 110, then the patient's heart is monitored to determine whether a subsequent defibrillation shock is necessary. If so, the above steps may be repeated to deliver the subsequent defibrillation shock.

FIG. 5 is a flow chart illustrating the operation steps of delivering an artifact-compensated defibrillation shock according to another embodiment of the present invention.

In operation, an instruction to stop the CPR operation is given to the rescuer in step 200, then the charging of the capacitor 44 to a full level is initiated. A short interval is allowed after the discontinuation of the CPR compression in step 202, then the electrodes 22 and 24 connected to the ECG front end 32 detect an input signal, i.e., ventricular fibrillation (VF) and the patient impedance, as well as the input signal due to potentially corrupted signal and/or the movement of the patient during a CPR operation in step 204.

The ECG and disturbance signals are analyzed, and if artifact/and or motion disturbance is detected in step 206, the process returns to step 204. If the ECG data is not corrupted, the ECG data is analyzed by processor 40 to determine if a shock is required in step 209. If a shock is not required, the AED may provide alternate care instructions to the rescuer in step 210. Note that absence of either ECG corruption or motion disturbance signals in step 108 indicates that the rescuer has discontinued CPR, and it is therefore appropriate to immediately arm the defibrillator to deliver a shock. If the ECG rhythm should be shocked, the AED completes its capacitor charge in step 208. After the capacitor 44 is charged fully, the processor 40 sends a notification signal to the user prior to delivering the defibrillation shock. In this way, the defibrillation shock is discharged to the patient, then the patient's heart is monitored to determine whether a subsequent defibrillation shock is necessary.

FIG. 6 is a flow chart illustrating the operation steps of delivering a defibrillation shock according to yet another embodiment of the present invention.

In this case, processor 40 monitors a CPR interval of a predetermined time. In step 300, as the end of the CPR interval approaches, processor 40 commands the charger 46 to fully charge capacitor 44 in a manner that full charge will be reached prior to the timeout of the CPR interval. Again, prior to the end of the CPR interval, ECG data plus the various signals indicative of disturbance are acquired in step 302. In step 304 the ECG data is examined for corruption. If the ECG data is corrupted due to CPR, more data is acquired. However, if the data is not corrupted, a determination is made if the patient's ECG rhythm should receive a defibrillation shock in step 306. In step 308, processor 40 continues to update the rhythm's shock determination until timeout of the CPR interval. Upon timeout of the CPR interval, in step 310 processor 40 causes voice circuit and speaker 41 to prompt the rescuer not to touch the patient. In step 312, processor 40 examines the recently determined shock decision. If a defibrillation shock is not appropriate, the device directs the rescuer to alternative patient care such as, for example, additional CPR. If a shock is appropriate, step 316 examines disturbance data to assure that detectable handling of the patient has ceased. If handling is detected, a prompt is issued to the rescuer to stop touching the patient per step 318. If no handling is detected, processor 40 arms the defibrillator to shock in step 320, and the AED immediately issues a prompt to the rescuer to deliver the shock. The advantage of this embodiment is that if the performance of CPR does not corrupt the patient's ECG signal, it is possible to make all of the needed decisions for the next shock while CPR is still ongoing. In this manner, the AED can be ready to deliver a shock immediately upon timeout of the CPR interval. Thus, the delay between cessation of CPR and the shock can approach zero seconds.

FIG. 7 is a flow chart illustrating the operation steps of delivering a defibrillation shock according to another embodiment of the present invention.

The electrodes 22 and 24 connected to the ECG front end 32 (FIG. 2) detect an input signal, for example, ventricular fibrillation (VF) and the patient impedance, measured by measuring a low-level test signal or delivering a non-therapeutic signal in step 702. Additionally, an input signal due to potentially corrupted signal and/or the movement of the patient is acquired. The motion information is obtained by motion disturbance detector 10 or motion sensor. At step 704, a determination is made whether the ECG data is corrupted. If the ECG data is determined to be corrupted, more data is acquired until the processor 40 can make a determination whether a shock is appropriate.

At step 708 the ECG data acquired at 702 is analyzed by the processor 40 to determine if a shock is required. A shock may be appropriate under certain conditions where a shockable rhythm is detected, for example, where the ECG data indicates the presence of VF or ventricular tachycardia (VT), as well known in the art. The processor 40 can utilize analyses previously described, or known in the art, to make the determination of whether a shock is appropriate. Following the determination if a shock is appropriate, the processor 40 initiates a CPR interval and sends signals to the voice circuit and speaker 41 or to the display 42, or to both, at step 706 to provide a visual and/or audio cue to the rescuer to begin CPR compressions. In an alternative embodiment, the steps 706 and 708 are reversed so that the CPR interval and administration of CPR are initiated prior to the determination if a shock is appropriate. In either order, the administration of CPR and the determination if a shock is appropriate can also overlap. At step 710 the voltage charger 46 begins to charge the energy storage capacitor network 44 at a time such that a full charge will be reached prior to the completion of the CPR interval. Upon the completion of the CPR interval, shown in FIG. 7 at step 712, the processor 40 sends a signal to the voice circuit and speaker 41 or to the display 42, or to both, directing the rescuer to cease administration of CPR and to stand clear of the patient.

A determination is made at step 714 whether the analysis performed by the processor 40 at the step 708 results in the administration of a defibrillating shock. In the event a shock is not appropriate, the AED may provide alternative care recommendations to the rescuer in step 715. In contrast, where a shock is determined to be appropriate, at step 716 the processor 40 examines disturbance data to assure that detectable handling of the patient has ceased. Where the disturbance data indicates that handling has not ceased, the processor 40 sends signals to the voice circuit and speaker 41 or to the display 42, or to both at step 718, to issue a prompt to the rescuer to stop touching the patient. When no disturbance is detected by the processor 40, the processor 40 arms the defibrillator 20 to deliver a defibrillating shock to the patient at step 720.

Following a process such as that described with respect to FIG. 7 allows for delivery of a defibrillation shock almost immediately following the completion of the CPR interval where a shockable rhythm is detected from the ECG signal acquired prior to the initiation of the CPR interval. In this manner, the time between the cessation of CPR compressions and delivery of the defibrillating shock can be reduced. The previously described process can be utilized with a variety of different resuscitation protocols.

While the preferred embodiments of the present invention have been illustrated and described, it will be understood by those skilled in the art that various changes and modifications may be made and equivalents may be substituted for elements thereof without departing from the true scope of the present invention. In addition, many modifications may be made to adapt to a particular situation and the teaching of the present invention without departing from the central scope. For example, the acquisition of the ECG signal for analysis can be performed without the determination of whether the ECG data is corrupted, as in step 704, and remain within the scope of the present invention. Therefore, it is intended that the present invention not be limited to the particular embodiment disclosed as the best mode contemplated for carrying out the present invention, but that the present invention include all embodiments falling within the scope of the appended claims.

What is claimed is:

1. A method for delivering a defibrillation shock to a patient from a defibrillator, the method comprising:
acquiring electrocardiographic (ECG) data representative of an ECG signal for the patient;
analyzing the ECG data to determine whether administration of a defibrillating shock is appropriate;
initiating a cardio-pulmonary resuscitation (CPR) interval subsequent to the acquisition of ECG data, CPR compressions administered to the patient for at least a portion of the CPR interval; and
in response to the completion of the CPR interval and if administration of a defibrillating shock is determined to be appropriate from the ECG data, arming the AED for delivery of the defibrillation shock to the patient.

2. The method of claim 1 wherein analyzing the ECG data to determine whether administration of a defibrillating shock is appropriate is completed before the CPR interval is concluded.

3. The method of claim 1 wherein analyzing the ECG data to determine whether administration of the defibrillating shock is appropriate comprises detecting at least one of ventricular fibrillation and ventricular tachycardia from the ECG data.

4. The method of claim 1 wherein acquiring ECG data representative of an ECG signal comprises:
obtaining an ECG signal;
determining whether the ECG signal is corrupted by movement of the patient; and
obtaining another ECG signal until an uncorrupted signal is acquired.

5. The method of claim 1, further comprising delivering the defibrillating shock to the patient subsequent to arming of the AED.

6. The method of claim 1, further comprising detecting an indication of CPR cessation prior to arming the AED.

7. The method of claim 1, further comprising providing alternative care recommendations in response to the completion of the CPR interval and if administration of a defibrillating shock is determined to be inappropriate.

8. A method for timing the delivery of a defibrillating shock from an automatic external defibrillator (AED), the method comprising:
acquiring an ECG signal prior to initiation of a CPR interval;
prior to the completion of the CPR interval, determining whether delivery of a defibrillating shock is appropriate; and
arming the AED for delivery of a defibrillating shock upon completion of the CPR interval if delivery of the defibrillating shock is determined to be appropriate,
otherwise, delaying AED arming and acquiring and analyzing an ECG signal upon completion of the CPR interval if delivery of the defibrillating shock is determined, based upon an ECG signal acquired prior to the CPR interval, to be inappropriate.

9. The method of claim 8 wherein analyzing the ECG signal prior to initiation of a CPR interval comprises analyzing an ECG signal subsequent to completion of a previous CPR interval.

10. The method of claim 8 wherein analyzing the ECG signal prior to initiation of a CPR interval comprises analyzing an ECG signal immediately prior to the initiation of a CPR interval.

11. The method of claim 8 wherein determining whether delivery of a defibrillating shock is appropriate comprises detecting at least one of ventricular fibrillation and ventricular tachycardia from the ECG signal analysis.

12. The method of claim 8, further comprising fully charging a charging capacitor prior to the completion of the CPR interval.

13. The method of claim 8, further comprising detecting an indication of CPR cessation prior to arming the AED and wherein arming the AED for delivery of the defibrillating shock occurs immediately after CPR cessation is detected.

14. The method of claim 8, further comprising triggering the AED to deliver the defibrillating shock to a patient subsequent to arming of the AED.

15. A defibrillator for delivering a defibrillating shock, the defibrillator comprising:
an energy delivery unit configured to provide a defibrillating shock after being armed;
an electrocardiographic (ECG) analysis unit configured to acquire an ECG signal and analyze the same, the ECG analysis unit generating a shock appropriate signal in response to determining that delivery of a defibrillating shock is appropriate based on the ECG signal analysis;
a cardiopulmonary resuscitation (CPR) interval timer coupled to the ECG analysis unit and configured to initiate a CPR interval after acquisition of the ECG signal and generate a time-out signal in response to the completion of the CPR interval; and
an arming circuit coupled to the ECG analysis unit, the CPR interval timer, and the energy delivery unit and configured to arm the energy delivery unit to deliver the defibrillating shock in response to receiving the shock appropriate signal and the time-out signal.

16. The defibrillator of claim 15, further comprising a charging circuit coupled to the energy delivery unit to fully charge the energy delivery unit prior to the completion of the CPR interval.

17. The defibrillator of claim 15, further comprising a detector circuit coupled to the arming circuit to enable the arming circuit to arm the energy delivery unit in response to detecting cessation of CPR activity.

18. The defibrillator of claim 15 wherein the ECG analysis unit is configured to generate the shock appropriate signal responsive to detecting at least one of ventricular fibrillation and ventricular tachycardia from the ECG signal.

19. The defibrillator of claim 15 wherein the ECG analysis unit comprises an ECG signal acquisition unit configured to obtain an ECG signal, determine whether the ECG signal is corrupted by movement, and obtain another ECG signal until an uncorrupted ECG signal is acquired.

20. The defibrillator of claim 15 wherein the ECG analysis unit is configured to complete analysis of the ECG signal prior to generating the shock appropriate signal.
